(19) United States
(12) Patent Application Publication
(10) Pub. No.: US 2009/0130036 A1
(43) Pub. Date: May 21, 2009

(54) SELF TANNING PRODUCT HAVING SLIMMING, FIRMING AND TONING PROPERTIES ASSOCIATED THEREWITH

(75) Inventors: Tatiana Kelly, Bedford, TX (US); Mary E. Cooper, Oklahoma City, OK (US)

Correspondence Address: DUNLAP CODDING, P.C., PO BOX 16370, OKLAHOMA CITY, OK, 73113, US

(73) Assignee: Goodier Cosmetics, L.L.C.

(21) Appl. No.: 12/354,361

(22) Filed: Jan. 15, 2009

Related U.S. Application Data
(62) Division of application No. 11/702,376, filed on Feb. 5, 2007.
(60) Provisional application No. 60/765,324, filed on Feb. 3, 2006.

(51) Int. Cl. A61K 8/30; A61Q 17/04

(52) U.S. Cl. 424/59

(57) ABSTRACT

A sunless or self-tanning composition having slimming, firming and toning properties associated therewith. The composition is an emulsion containing safe effective amounts of sunless tanning agents and slimming, firming and toning constituents. A method for preparing such composition is disclosed.

### CROSS REFERENCE TO RELATED APPLICATIONS

This application is a divisional of U.S. Ser. No. 11/702,376, filed Feb. 5, 2007, which claims benefit under 35 U.S.C. 119(e) of U.S. Ser. No. 60/765,324, filed Feb. 3, 2006. Each of the above referenced patent applications are hereby expressly incorporated herein by reference in their entirety.

### BACKGROUND OF THE INVENTION

1. Field of the Invention

The present invention relates generally to self-tanning compositions, and more particularly to self-tanning compositions having slimming, firming and toning properties associated therewith.

2. Brief Description of the Prior Art

Many people use self-tanning compositions to achieve the look of a suntan without exposure to the sun, to obtain a darker suntan or to extend the natural life of a suntan. Most self-tanning compositions affect only the stratum corneum or outermost layer of the epidermis.

Tans produced by the prior art sunless or self-tanning compositions have encountered several problems. For example, tans produced by the prior art sunless or self-tanning compositions generally fade within 3 to 5 days due to the sloughing off or wearing away of dead skin cells from the surface of the skin. For this reason, it is generally desirable to reapply the sunless or self-tanning composition frequently to maintain the tan appearance. In addition, problems may arise in using sunless or self-tanning compositions because of the difficulty in obtaining uniform application of the sunless or self-tanning compositions, and the time required for such compositions to dry.

Further, many people desire compositions capable of slimming, firming and toning the body. It would be highly desirable if one could utilize a sunless or self-tanning composition having slimming, firming and toning properties associated therewith such that upon application of the sunless or self-tanning composition one could not only obtain a desired tan, but the fat cells of the body could also be dehydrated thereby reducing their size which results in slimming, firming and toning of the body.

It is to such sunless or self-tanning compositions having slimming, firming and toning properties associated therewith that the present invention is directed, as well as method for producing such sunless or self-tanning compositions.

### SUMMARY OF THE INVENTION

Broadly, the present invention relates to an emulsified sunless or self-tanning composition formulated from a water phase and an oil phase wherein the compositions, in addition to self-tanning or sunless tanning properties, also have slimming, firming and toning properties associated therewith. The water phase contains at least one solvent, such as water, at least one humectant, at least one phosphodiesterase inhibitor which functions as a slimming and toning agent, and at least one emulsion stabilizer or viscosity control agent, and at least one chelating agent. The oil phase contains at least one additional viscosity control agent, hydrocarbon solvent, emulsifying agent, emollient, emulsion stabilizer, buffering agent, skin coloring/self-tanning agent, humectant, a lipolytic enzyme activator, an adipogenesis and lipogenesis inhibitor, a surfactant, antioxidant, a catabolism enhancer (i.e., a firming agent), a cytokin production stimulation, a film former, a fragrance or masking agent and preservative.

### DETAILED DESCRIPTION OF THE INVENTION

As previously stated, the present invention relates to a sunless or self-tanning composition formulated of a water phase and an oil phase, the sunless or self-tanning compositions also having slimming and toning and/or firming properties associated therewith. The oil phase, which is admixed with the water phase in a plurality of stages, contains an effective amount of sunless or self-tanning agents to provide the resulting composition with the desired self-tanning properties, and an effective amount of ingredients containing slimming, toning and/or firming properties to provide the sunless or self-tanning composition with desired slimming, toning and/or firming properties.

In formulating the emulsified sunless or self-tanning compositions of the present invention having slimming, toning and/or firming properties associated therewith, the water phase contains water, a humectant, an effective amount of at least one slimming and toning agent (i.e., a phosphodiesterase inhibitor), to dehydrate fat cells of the body and thereby reduce the size of the fat cells which results in slimming, firming and toning of the body, and minor effective amounts of an emulsion stabilizer or viscosity control agent and a chelating agent.

While the amount of each constituent employed in the formulation of the water phase of the composition of the present invention can vary widely, generally the water phase contains water, from about 0.5 to about 50 weight percent of a humectant, based on the total weight of the composition, from about 0.01 to about 10 weight percent of at least one slimming, toning and firming agent (i.e., a phosphodiesterase inhibitor) based on the total weight of the composition, from about 0.05 to about 1 weight percent of an emulsion stabilizer or viscosity control agent, based on the total weight of the composition, and from about 0.05 to about 2 weight percent of a chelating agent, based on the total weight of the composition.

The amount of each constituent employed in the formulation of the various phases of the oil phase of the composition of the present invention which can vary widely. However, the oil phase of the composition will generally contain from about 0.62 to about 85 weight percent of a solvent, based on the total weight of the composition, from about 0.07 to about 30 weight percent of self-tanning or sunless tanning agents, based on the total weight of the composition, safe and effective amounts of slimming, toning and/or firming agents (i.e., from about 0.01 to about 25 weight percent of slimming, toning and/or firming agent), from about 0.65 to about 65 weight percent skin conditioners, based on the total weight of the composition, from about 0.4 to about 80 weight percent of emollients, based on the total weight of the composition, and from about 0.01 to about 10 weight percent film forms, based on the total weight of the composition. In addition, the various phases of the oil phase of the composition of the present invention include minor effective amounts of chelating agents and fragrances, the fragrances providing the composition formed by the admixture of the water phase and the various phases of the oil phase with a pleasing aroma.

As will be described in more detail hereinafter, the sunless or self-tanning compositions are emulsified compositions formulated by admixing various phases of the oil phase with a water phase. The constituent of the water phase and each of the various phases of the oil phase are as follows:

| | | Operable % | | |
|---|---|---|---|---|
| Phase | INCI Name | w/w (Range)⁽¹⁾ | Chemical | Function |
| A | Water | qs to 94 | Inorganic | Solvent |
| A | PEG-8 | 0.50-50.00 | Polymeric | Humectant |
| | | | Ether | |
| A | Caffeine | 0.01-10.00 | Heterocyclic | Slimming and Toning |
| | | | Compound | Agent |
| A | Xanthan Gum | 0.05-1.00 | Gum | Emulsion |
| | | | | Stabilizer |
| A | Tetrasodium | 0.05-2.00 | Alkyl Substituted | Chelating |
| | EDTA | | Amino Acid Salt | Agent |
| B | Glyceryl Stearate | 0.50-15.00 | Ester | Emulsifying Agent |
| B | Simmondsia Chinensis | 0.10-20.00 | Ester | Emollient |
| | (Jojoba) Seed Oil | | | |
| B | Ethylhexyl Stearate | 0.10-20.00 | Ester | Emollient |
| B | Dimethicone | 0.10-20.00 | Siloxane | Emollient |
| B | Cetyl Esters | 0.05-5.00 | Wax | Viscosity Controlling |
| | | | | Agent |
| B | Cyclopenta-Siloxane | 0.10-20.00 | Siloxane | Emollient |
| B | Ceteryl Alcohol | 0.05-10.00 | Fatty Alcohol | Emulsion Stabilizer |
| B | PEG-100 Stearate | 0.10-10.00 | Alkoyxylated | Emulsifying Agent |
| | | | Carboxcylic Acid | |
| B | Ceteareth-20 | 0.01-10.00 | Alkoxylated | Emulsifying Agent |
| | | | Alcohol | |
| C | Poly-Acrylamide | 0.01-10.00 | Synthetic Amide | Viscosity Controlling |
| | | | Polymer | Agent |
| C | C13-14 Isoparaffin | 0.01-10.00 | Hydrocarbon | Solvent |
| C | Laureth-7 | 0.05-3.00 | Alkoxylated | Emulsifying Agent |
| | | | Alcohol | |
| D | Lactic Acid | 0.01-10.00 | Carboxcyclic | Buffering Agent |
| D | Triethanol-Amine | 0.01-10.00 | Alkanol-Amine | Buffering Agent |
| E | Ethoxy-Diglycol | 0.10-25.00 | Alcohol Ether | Solvent |
| E | Dihydroxy-Acetone | 0.01-10.00 | Alcohol Ketone | Self-Tanning Agent |
| F | Denatured Alcohol | 0.50-25.00 | Alcohol | Solvent |
| F | Glycerin | 0.10-10.00 | Polyol | Humectant |
| F | Erythrulose | 0.01-10.00 | Alcohol Ketone | Self-tanning Agent |
| F | Bupleurum Falcatum | 0.01-10.00 | Botanical | Lipolytic Enzyme |
| | Root Extract | | | Activator-Slimming |
| | | | | Agent |
| F | Juglans Regia | 0.05-10.00 | Botanical | Self-Tanning Agent |
| | (Walnut) Extract | | | |
| F | Sodium Hyaluronate | 0.05-5.00 | Biological | Humectant |
| | | | Polymer | |
| F | Dihydro-Myricetin | 0.001-5.000 | Flavanone | Adipogenesis and |
| | | | | Lipogenesis Inhibitor- |
| | | | | Slimming Agent |
| F | Steareth-20 | 0.01-10.00 | Alkoxylated | Surfactant |
| | | | Alcohol | |
| F | Tocopherol | 0.001-5.000 | Heterocyclic | Antioxidant |
| | | | Compound | |
| F | Coenzyme A | 0.000001-5.000000 | Heterocyclic | Catabolism Enhancer- |
| | | | Thio Compound | Slimming Agent |
| F | Palmitoyl Tetrapeptide-7 | 0.000001-5.000000 | Protein | Cytokine Production |
| | | | | Stimulator-Firming |
| | | | | Agent |
| F | Trimethyl-Pentanediol/ | 0.01-10.00 | Synthetic | Film Former |
| | Adipic Acid Copolymer | | Polymer | |
| F | Propylene Glycol | 0.01-25.00 | Polyol | Solvent |
| F | Fragrance | 0.01-10.00 | | Fragrance |
| F | Diazolidinyl Urea | 0.05-1.50 | Heterocyclic | Preservative |
| | | | Amide | |
| F | Methyl-Paraben | 0.05-1.00 | Phenolic Ester | Preservative |
| F | Propyl-Paraben | 0.05-1.00 | Phenolic Ester | Preservative |
| F | Iodopropynyl Butyl- | 0.001-0.050 | Halogenated | Preservative |
| | Carbamate | | Amide Ester | |

⁽¹⁾An abbreviation for “by weight” used in chemistry and pharmacology to describe the concentration of a substance in a mixture or solution. For example, 30.00 to 85.00 w/w means that the mass of the substance is 30.00 to 85.00 percent of the total mass of the solution or mixture.

From the above, it becomes apparent that the sunless or self-tanning compositions having slimming, toning and/or firming properties of the present invention formulated from the above-described water phase and the various phases of the oil phase contain from about qs to 94 weight percent water, based on the total weight of the composition, from about 0.07 to about 30 weight percent of self-tanning agents, based on the total weight of the composition, safe and effective amounts of slimming, toning and/or firming agents (i.e., from about 0.02% to about 35 weight percent of the slimming, toning, and/or firming agents), from about 0.65 to about 65 weight percent skin conditioners, based on the total weight of the composition, from about 0.4 to about 80 weight percent of emollients and from about 0.01 to about 10 weight percent of film formers, based on the total weight of the composition. In addition, the compositions further include minor effective amounts of pH adjusters which provide the compositions with a pH in the range of from about 3.25 to about 3.75, chelating agents and fragrances to provide the composition with a pleasing aroma. In each of the above ingredients it is important that a safe and effective amount of the ingredients be incorporated into the various phases of the formulation to achieve the desired effects of such constituents.

The term “safe and effective amount” is used herein means that the amount of a compound or composition is sufficient to significantly induce a positive benefit, including independently or in combination the benefits disclosed herein, but low enough to avoid serious side effects, (i.e., to provide a reasonable benefit to risk ratio, within the scope of sound judgment of the skilled artisan).

The self-tanning or skin coloring agents used in the formulation of the compositions of the present invention can be any skin coloring agent compatible with the other ingredients of the composition. Examples of such self-tanning agents which may be employed in the practice of the present invention include, but are not limited to, dihydroxy acetone, erythrulose, melanin, mahakanni, walnut extract, combinations thereof and the like.

The constituents providing the desired slimming and toning properties of the compositions of the present invention can vary widely, the only requirement being that such constituents are compatible with the other constituents or ingredients of the compositions of the present invention, and that such constituents are capable of dehydrating and thus reducing the size of the fat cells of the skin to which the compositions are applied. Examples of constituents which can be employed to provide the desired slimming and toning properties to the compositions of the present invention are caffeine, lipolytic enzyme activator such as bupleurum falcatum root extract, dihydromyricetin, catabolism enhancers such as coenzyme A, hypnea musciformis, gellididela acerosa, sargassum filipdendula, spirulina and the like.

Similarly, the constituents which provide the composition with the desired firming properties can also vary widely and can be any cytokine production stimulator compatible with the other constituents of the composition, such as palmitoyl tetra peptide-7.

The term “film former” as used herein includes compounds for aiding the film forming properties of the composition. Further, such compounds can be dissolved or disposed in water to form a system of increased viscosity and which upon evaporation of the water in the system leaves a coherent film. Examples of film formers which can be used in the formulation of the composition can be any well known compounds that are compatible with the self-tanning or sunless tanning agents and the slimming, firming and toning agents of the compositions as well as the other ingredients of such compositions. Examples of such film formers are xanthan gum, guar gum, synthetic polymers such as Trimethylpentanediol, adipic acid copolymer, hydroxyethyl cellulose, polyvinyl alcohol and the like.

The term “chelating agent” as used herein is to be understood to mean an active agent capable of removing a metal from a composition by forming a complex so that the metal ion cannot readily participate in or catalyze chemical reactions. The inclusion of a chelating agent is especially useful for providing protection against UV radiation which can contribute to excessive scaling or skin texture agents or against other environmental agents which can cause skin damage.

The term “skin conditioners” as used herein include humectants, moisturizers, skin conditioners, and the like. These materials include, but are not limited to, guanidine; urea; glycolic acid and glycolate salts (e.g., ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, gucosamine); hyaluronic acid, lactamide monoethanolamine; acetamide monoethanolamine; panthenol; allantoin; and mixtures thereof.

The terms “viscosity controlling agents”, “emulsifier”, “emulsifying agents”, and “emulsion stabilizer(s)”, as used herein function as thickening and gelling agents and include, but are not limited to, gums, such as, acacia, agar, algin, alginic acid, ammonium alignate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof. Other examples of emulsion stabilizers are ceteareth-20, PEG-100 stearate, metallic chloride, metallic chlorohydroxide, phosphoric acid, beeswax, clays, cetyl alcohol, lanolin, glyceryl stearate, and polysorbates, sodium carbomer, sodium chloride and stearic alcohol.

Preferred compositions of the present invention include a thickening agent selected from carboxylic acid polymers, crosslinked polyacrylate polymers, polyacrylamide polymers, and mixtures thereof, more preferably thickening agents selected from carboxylic acid polymers, polyacrylamide polymers and mixtures thereof.

The term “emollient” as used herein refers to a material useful for the prevention or relief of dryness, as well as for the protection of the skin. A wide variety of suitable emollients are known and may be used herein. Examples of such “emollients” are simmondisia chinensis (jojoba) oil, ethylhexyl stearate, dimethicone, and cyclophentasiloxane.

The term “solvents” as used herein is to be understood to mean a substance in which another substance is dissolved, forming a solution. Solvents are used as a base medium for cosmetic preparations. Examples of such solvents are hexane, benzene, toluene, acetone, acetonitrile, acetic acid, n-butanol, methanol, formic acid and water.

The term “buffering agent” as used herein is to be understood to mean a substance that maintains the pH of an aqueous medium in a narrow range even if small amounts of acids or bases are added. Examples of such buffering agents are calcium carbonate and sodium bicarbonate.

The term “lipolytic enzyme activator” as used herein is to be a substance whose general activity is to hydrolyse fats present in the ester form (such as the glycerides found in human skin), and accordingly generate fatty acids and glycerol. An example of a lipolytic enzyme activator is bupleurum falcatum root extract.

The term “adipogenesis/lipogenesis inhibitor as used herein is to be understood to mean a substance which prevents the production of fatty acids. An example of an adipogenesis/lipogenesis inhibitor is flavanone.

The term “surfactant” as used herein is to be understood to mean a surface-active compound containing both lipophile and hydrophile groups which facilitate the cleansing of skin and hair. Examples of such surfactants are cocamidopropyl betaine, coco glucoside, and sodium laureth sulfate.

The term “antioxidant” as used herein is to be understood to mean a substance used to prevent or slow deterioration from reaction with oxygen. Examples of such antioxidants are ascorbic acid, BHT, green tea extract, rosemary extract and tocopherol.

The term “cytokine production stimulator” as used herein is to be understood to mean a substance which enhances collagen biosynthesis, improves cell repair, supports the skin immune system, and improves the skin barrier. Examples of such a cytokine production stimulator is palmitoyl tetrapeptide-7, Heather extract, and Pyrus Malus extract.

The term “fragrance or masking agent” as used herein is to be understood to mean a substance designed to emit a pleasant odor and cover an undesirable odor. An example of such a fragrance is rosemary.

The term “preservative” as used herein is to be understood to mean a substance which prevents or retards microbial growth and protects a product from spoilage. Examples of such a preservative are DMDM hydantoin, imidazolidinyl urea, methylpanaben, potassium sorbate, propylparaben, and sodium benzoate.

In formulating the sunless or self-tanning compositions having slimming, toning and/or firming properties of the present invention, the constituents of phase A (i.e., the water phase) of the formulation are blended together at a temperature of about 65° C. to about 80° C. Once a uniform aqueous mixture has been formed of the constituents of phase A, the ingredients of phase B (i.e., the initial oil phase) are admixed while heating at a temperature of about 65° C. to about 80° C. under vigorous stirring to form a stable oil-in-water emulsion. Once the initial oil-in-water emulsion is formed from the constituent of phase A and B, the initial oil-in-water emulsion is allowed to cool to a temperature of from about 35° C. to about 55° C. at which time the constituents of phase C are admixed into the initial oil-in-water emulsion formed by admixing the constituents of phase A and phase B. Once a substantially uniform emulsion is formed containing the constituents of phases A, B, and C, each constituent of phase D is added individually to the emulsion, (i.e., the constituents of phase D are not premixed). In addition, the ingredients of phase D are introduced into the emulsion formed from the constituents of phases A, B, and C at a temperature in the range of from about 35° C. to about 55° C. Once the constituents of phase D have been added, the constituents of phase E are admixed separately into the oil-in-water emulsion formed from the constituents of phases A-D. Therefore, the constituents of phase E are introduced into the oil-in-water emulsion formed from the constituents of phases A, B, C and D. The admixing of the constituents of phase E with the oil-in-water emulsion formed from admixing the constituents of phases A, B, C and D is also carried out at a temperature of from about 35° C. to about 55° C. Lastly, the constituents of phase F are added to the oil-in-water emulsion produced from admixing the constituents of phases A, B, C, D and E and the resulting oil-in-water emulsion is mixed at room temperature for a period of time effective to provide a substantially uniform stable oil-in-water emulsion, i.e., the sunless or self-tanning composition having slimming, firming and toning properties associated therewith of the present invention.

In order to more fully describe the present invention, the following example is set forth. However, it is to be understood that the example is for illustrative purposes only and is not to be construed as limiting the invention disclosed herein.

### EXAMPLE

| | | Actual % | |
|---|---|---|---|
| Phase | INCI Name | w/w⁽¹⁾ | Function |
| A | Water | 66.5523 | Solvent |
| A | PEG-8 (Polymeric Ether) | 4.9000 | Humectant |
| A | Caffeine | 0.2800 | Phospho-Diesterase |
| | | | Inhibitor-Slimming Agent |
| A | Xanthan Gum | 0.1000 | Emulsion |
| | | | Stabilizer |
| A | Tetrasodium | 0.2000 | Chelating Agent |
| | EDTA | | |
| B | Glyceryl Stearate | 3.2500 | Emulsifying Agent |
| B | Simmondsia Chinensis | 3.0000 | Emollient |
| | (Jojoba) Seed Oil | | |
| B | Ethylhexyl Stearate | 3.0000 | Emollient |
| B | Dimethicone | 2.0000 | Emollient |
| B | Cetyl Esters | 0.5000 | Viscosity Controlling Agent |
| B | Cyclopenta-Siloxane | 1.0000 | Emollient |
| B | Ceteryl Alcohol | 0.8200 | Emulsion Stabilizer |
| B | PEG-100 Stearate | 0.7500 | Emulsifying Agent |
| B | Ceteareth-20 | 0.1800 | Emulsifying Agent |
| C | Poly-Acrylamide | 0.8000 | Viscosity Controlling Agent |
| C | C13-14 Isoparaffin | 0.4000 | Solvent |
| C | Laureth-7 | 0.1100 | Emulsifying Agent |
| D | Lactic Acid | 1.0000 | Buffering Agent |
| D | Triethanol-Amine | 0.4300 | Buffering Agent |
| E | Ethoxy-Diglycol | 2.0000 | Solvent |
| E | Dihydroxy-Acetone | 0.5000 | Self-Tanning Agent |
| F | Butylene Glycol | 2.2300 | Solvent |
| F | Denatured Alcohol | 2.000 | Solvent |
| F | Glycerin | 0.4500 | Humectant |
| F | Erythrulose | 0.5000 | Self-Tanning Agent |
| F | Bupleurum Falcatum | 0.2100 | Lipolytic Enzyme |
| | Root Extract | | Activator-Slimming |
| | | | Agent |
| F | Juglans Regia (Walnut) | 0.3300 | Self-tanning Agent |
| | Extract | | |
| F | Sodium Hyaluronate | 0.1000 | Humectant |
| F | Dihydro-Myricetin | 0.0360 | Adipogenesis and |
| | | | Lipogenesis Inhibitor- |
| | | | Slimming Agent |
| F | Steareth-20 | 0.0900 | Surfactant |
| F | Tocopherol | 0.1000 | Antioxidant |
| F | Coenzyme A | 3 ppm | Catabolism Enhancer- |
| | | | Slimming Agent |
| F | Palmitoyl Tetrapeptide-3 | 14 ppm | Cytokine Production |
| | | | Stimulator-Firming |
| | | | Agent |
| F | Trimethyl-pentanediol/ | 1.0000 | Film former |
| | Adipic Acid Copolymer | | |
| F | Propylene Glycol | 0.3300 | Solvent |
| F | Fragrance | 0.2500 | Masking Agent |
| F | Diazolidinyl Urea | 0.1980 | Preservative |
| F | Methyl-Paraben | 0.2500 | Preservative |
| F | Propyl-Paraben | 0.1500 | Preservative |

⁽¹⁾An abbreviation for “by weight” used in chemistry and pharmacology to describe the concentration of a substance in a mixture or solution. For example, 30.00 to 85.00 w/w means that the mass of the substance is 30.00 to 85.00 percent of the total mass of the solution or mixture.

In formulating the sunless or self-tanning composition having slimming, firming and toning properties, the constituents of phase A are blended together at a temperature of about 75° C. Once a substantially uniform mixture has been formed of the constituents of phase A, the ingredients of phase B are admixed with the mixture formed of the constituents of phase A, while heating at a temperature of about 75° C., under vigorous stirring to form a stable oil-in-water emulsion. Once the oil-in-water emulsion is formed from the constituent of phase A and B, the oil-in-water emulsion is allowed to cool to a temperature of about 75° C. at which time the constituents of phase C are admixed into the oil-in-water emulsion formed by admixing the constituents of phase A and phase B. Once a substantially uniform emulsion is formed containing the constituents of phases A, B, and C, each constituent of phase D is added individually to the emulsion, (i.e., the constituents of phase D are not premixed). In addition, the ingredients of phase D are introduced into the emulsion formed from the constituents of phases A, B, and C at a temperature of about 75° C.

Once the constituents of phase D have been added, the constituents of phase E are each added and admixed separately with the oil-in-water emulsion formed from the constituents of phases A-D. Therefore, the constituents of phase E are introduced into the oil-in-water emulsion formed from the constituents of phases A, B, C and D. The admixing of the constituents of phase E with the oil-in-water emulsion formed from admixing the constituents of phases A, B, C and D is also carried out at a temperature of about 65° C. Lastly, the constituents of phase F are added to the oil-in-water emulsion produced from admixing the constituents of phases A, B, C, D and E and the resulting oil-in-water emulsion is mixed at room temperature for a period of time effective to provide the sunless or self-tanning composition having slimming, firming and toning properties of the present invention which is a substantially uniform state oil-in-water emulsion.

The self-tanning compositions having slimming, firming and toning properties associated therewith of the present invention represent an improvement in the art of sunless self-tanning compositions because the compositions of the present invention:
Contains a combination of Dihydroxyacetone and Erythrulose for a natural, even tan color.
Contains a combination of clinically tested ingredients which reduce fat (adipose) tissue and which firm and tighten the skin.
   Previously slimming or cellulite products only worked to reduce the appearance of fat.
   The compositions of the present invention contain ingredients which firm the skin which is known to become more slack with age.
   After major weight loss, a person usually must have loose skin surgically removed. The concept of including an ingredient that firms the skin is similar. After adipose (fat) tissue is reduced, skin must be firmed to improve appearance.
Contains clinical level of Lipocare (Bupleurum Falcatum Extract, CoEnzyme A, Caffeine) which stimulates the breakdown of fat tissue.
   Lipocare has been shown to reduce the appearance of cellulite (orange peel skin) by up to about 33%.
Bupleurum Falcatum Extract can be referred to a “Chinese herb”
Contains clinical levels of Rigin (Palmitoyl Tetrapeptide-3), an ingredient which has been shown to enhance the firmness and elasticity of the skin by up to about 39% and about 27%, respectively.
Results will normally be seen after 8 weeks when the compositions of the present invention are applied twice daily.

From the above, it is clear that the present invention is well adapted to carry out the objects and to attain the advantages mentioned herein as well as those inherent in the invention. While presently preferred embodiments of the invention have been described for purposes of this disclosure, it will be understood that numerous changes may be made which will readily suggest themselves to those skilled in the art and which are accomplished within the spirit of the invention disclosed and claimed.

What is claimed is:

1. A method for producing a self-tanning composition having slimming, firming and toning properties, the method comprising the steps of:
admixing water and safe and effective minor amounts of at least one humectant, at least one slimming and toning agent, at least one emulsion stabilizer and at least one chelating agent at a temperature of from about 65° C. to about 80° C. for a period of time effective to form a substantially uniform phase A aqueous mixture;
mixing effective amounts of at least one emollient, and at least one emulsifier at a temperature of from about 65° C. to about 80° C. for a period of time to provide a substantially uniform phase B mixture;
admixing the phase A aqueous mixture with the phase B mixture at a temperature of from about 65° C. to about 80° C. for a period of time effective to provide a substantially uniform mixture;
permitting the resulting mixture formed by admixing phases A and B to cool to a temperature of from about 35° C. to about 55° C.;
admixing effective amounts of a solvent, and at least one emulsifier to form a substantially uniform phase C mixture;
admixing the phase C mixture with the resulting mixture containing phases A and B at a temperature of from about 65° C. to about 80° C. from a period of time to form a substantially uniform mixture of phases A, B, and C;
cooling the substantially uniform mixture of phases A, B and C to a temperature of from about 35° C. to about 55° C.;
admixing at least one buffering agent constituting phase D with the substantially uniform mixture of phases A, B and C at a temperature of from about 35° C. to about 55° C. to form a substantially uniform mixture of phases A, B, C and D;
admixing effective amounts of a solvent, and a self-tanning agent to form a substantially uniform phase E mixture;
admixing the phase E mixture with the mixture containing phases A, B, C and D at a temperature of from about 35° C. to about 55° C. for a period of time to form a substantially uniform mixture containing the constituents of phases A, B, C and D;
permitting the mixture containing the mixture of phases A, B, C, D and E to cool to room temperature;
admixing at least one viscosity control agent, at least one hydrocarbon solvent, at least one emulsifying agent, at least one emollient, at least one buffering agent, at least one self-tanning agent, at least one humectant, at least one lipolytic enzyme activator, at least one lipogenesis inhibitor, at least one surfactant, at least one antioxidant, at least one catabolism enhancer, at least one cytosine production stimulator, at least one film former, at least one masking agent and at least one preservative at room temperature to provide a substantially uniform phase F mixture; and
admixing the phase F mixture with the phase A, B, C, D and E mixtures at room temperature to provide a self-tanning composition containing slimming, firming and toning properties.

2. The method of claim 1 wherein:
the phase A aqueous mixture contains from about 0 to about 94% w/w water, from about 0.50-50.00% w/w of at least one4 humectant, from about 0.01-10.00% w/w of at least one slimming and toning agent, from about 0.05-2.00% w/w of at least one chelating agent and from about 0.05-1.00% w/w of at lest one emulsion stabilizer;
the phase B mixture contains from about 0.10-20.00 emollients, and from about 0.01-10.00% w/w emulsifiers;
the phase C mixture contains from about 0.01-10.00% w/w of at least emulsifier and from about 0.01-10.00% w/w of the solvent;
the phase D mixture contains from about 0.01-10.00% w/w buffering agent;
the phase E mixture contains from about 0.10-25.00% w/w solvent, and from about 0.01-10.00% w/w self tanning agent; and
the phase F mixture contains from about 0.50-25.00% w/w of at least one hydrocarbon solvent, from about 0.01-10.00% w/w of at least one self-tanning agent, from about 0.10-10.00% w/w of at least one humectant, from about 0.01-10.00% w/w of at least one lipolytic enzyme activator, from about 0.0001-5.0% w/w of at least one lipogenesis inhibitor, from about 0.01-10.00% w/w of at least one surfactant, from about 0.001-5.0% w/w of at least one antioxidant, from about 0.000001-5.0% w/w of at least one catabolism enhancer, from about 0.000001-5.0% w/w of at least one cytosine production stimulator, from about 0.01-10.00% w/w of at least one film former, from about 0.01-10.00% w/w of at least one masking agent and from about 0.05-1.50% w/w of at least one preservative.

3. The method of claim 2 wherein the emulsion stabilizer is xanthan gum, the chelating agent is tetrasodium EDTA, the slimming and toning agent is caffeine, and the humectant is PEG-8.

4. The method of claim 2 wherein the at least one emollient is selected from the group consisting of simmondisia chinensis (jojoba) oil, ethylhexyl stearate, dimethicone, and cyclophentasiloxane, the at least one emulsifier is selected from a group consisting of ceteareth-20, PEG-100 stearate and glyceryl stearate, the film former is trimethylpentanediol, adipic acid copolymer and the preservative is propylparaben.

5. The method of claim 2 wherein the emulsifier is polyacrylamide, C13-14 isoparaffin, laureth-7.

6. The method of claim 2 wherein the pH adjusters are selected from a group consisting of lactic acid and triethanolamine.

7. The method of claim 2 wherein the base is deionized water, the solubilizer is ethoxydiglycol, and the self-tanning agent is dihydroxyacetone.

8. The method of claim 2 wherein the peptide is a mixture of glycerin, butylene glycol, carbomer, polysorbate-20, palmitoyl oligopeptide, palmitoyl tetrapeptide-7, the skin conditioners are selected from a group consisting of a mixture of water, propylene glycol, walnut extract and tocopherol, the self-tanning agent is erythrulose, the moisturizer is sodium hyaluronate, the preserveratives are selected from a group consisting of diazolidinyl urea and idopropynyl butyl carbamate.
